(19) United States
(12) Patent Application Publication
(10) Pub. No.: US 2012/0198578 A1
(43) Pub. Date: Aug. 2, 2012

(54) VIRAL ASSAY

(75) Inventors: John William Lowenthal, Belmont (AU); Timothy James Doran, Ocean Grove (AU); Scott Geoffrey Tyack, Grovedale (AU); Terry Glenn Wise, Waurn Ponds (AU); Scott McLeod, Midlothian (GB)

(73) Assignee: Commonwealth Scientific and Industrial Research Organisation, Campbell, Australian Capital Territor (AU)

(21) Appl. No.: 13/378,316

(22) PCT Filed: Jun. 18, 2010
(86) PCT No.: PCT/AU10/00752

§ 371 Date: Apr. 6, 2012

(51) Int. Cl. C12Q 1/70; C07H 21/04; A01K 67/02

(52) U.S. Cl. 800/13; 435/5; 536/24.33

(57) ABSTRACT

The present invention relates to an assay for detecting virus, in particular an assay for detecting viral replication in a tissue sample. The invention also relates to methods of determining the susceptibility of an animal to a virus, and methods of breeding animals with decreased susceptibility to a virus.

### FIELD OF THE INVENTION

The present invention relates to an assay for detecting virus, in particular an assay for detecting viral replication in a tissue sample. The invention also relates to methods of determining the susceptibility of an animal to a virus, and methods of breeding animals with decreased susceptibility to a virus.

### BACKGROUND OF THE INVENTION

Viral infection remains an important health problem in both humans and animals with adverse economic and social consequences. For example, there are a number of viral pathogens that cause disease in economically important livestock animals such as chickens, pigs, fish, sheep and cattle. Viral diseases of livestock animals include Avian Influenza, Newcastle Disease, Chicken Anaemia and Infectious Bursal Disease in chickens, Foot and Mouth Disease in cloven-hoofed animals, Porcine Reproductive and Respiratory Syndrome (PRRS) and Classical Swine Fever in pigs, Bluetongue and Akabane disease in sheep, and Infectious Salmon Anemia, Infectious Hematopoietic Necrosis Virus disease (IHNV), Viral Haemorrhagic Septicaemia and Infectious Pancreatic Necrosis in fish.

One of the main approaches to protecting animals from viral disease is vaccination. Vaccination of livestock in some circumstances is not commercially feasible due to the costs associated with the production and administration of vaccines. In addition, many vaccines do not provide complete protection and may make it difficult to distinguish between vaccinated and infected animals.

Selecting and breeding animals with decreased susceptibility to a virus could assist in developing animal stock with increased innate immunity to a viral pathogen and so may ultimately reduce the need for vaccination of commercial livestock. A major limitation of some current methods for determining the susceptibility of an animal to a virus, however, is that in order to obtain a suitable tissue sample, the animal needs to be euthanized. As a result, such methods cannot be used to select animals for breeding purposes. Another limitation of some current methods is that it is necessary to establish a cell culture line or cultivate tissue from an animal before the susceptibility of the animal to the virus can be determined. Such methods involving the establishment of cell or tissue culture are time consuming.

Another way in which to decrease the susceptibility of an animal to a virus may be via the insertion of a transgene into the animal to provide innate viral resistance. The viral resistance will persist throughout their lives and will be transmitted to their offspring. This viral resistance may be conferred by a transgene which expresses a double-stranded RNA (dsRNA) and so utilises RNA interference to provide innate immunity against a viral pathogen. In an alternative approach, the transgene may express a gene native to the animal species to which the host animal belongs and which may be, for example, a cytokine which increases the animals immunity to a viral pathogen. In such instances it would be desirable to be able to screen for transgenic animals which have a decreased susceptibility to a virus so that those animals may be used for breeding.

There remains a need for tests suitable for determining the susceptibility of animals to viral infection that can be performed on live animals. Such a test could be used, for example, to select animals with a decreased susceptibility to a virus for breeding purposes.

### SUMMARY OF THE INVENTION

The present inventors have now shown that viruses are able to replicate in tissue samples obtained from an animal and that detection of viral replication in the tissue samples may provide an indication of the susceptibility of the animal to infection.

Accordingly, the present invention provides a method for determining the susceptibility of a subject to a virus, the method comprising:

contacting a tissue sample obtained from the subject with the virus,

incubating the tissue sample for time sufficient for viral replication, and

detecting the presence or absence of virus in the tissue sample.

The present invention further provides a method for detecting viral replication in a tissue sample from a subject, the method comprising:

contacting a tissue sample obtained from the subject with a virus,

incubating the tissue sample for time sufficient for viral replication, and

detecting the presence or absence of virus in the tissue sample.

In one embodiment, the method further comprises removing virus that is not attached to a cell in the tissue sample prior to incubating the sample for time sufficient for viral replication.

In another embodiment, the presence of virus is indicative of susceptibility to the virus.

In yet another embodiment of the invention, the method further comprises comparing the level of virus in the sample with a control sample. The control sample may be a sample which contains a known level of virus, or a sample that does not contain any virus.

The method of the present invention may detect an increased or decreased level of virus in a sample compared to a control sample.

In one embodiment, a decreased level of virus in the tissue sample compared to the control sample is indicative of a decreased susceptibility to the virus.

While the methods of the invention may be performed on any suitable subject, in one particular embodiment the subject is avian, including poultry, for example, a chicken.

In another embodiment, the subject is a fish, for example, a salmonid. Preferably, the salmonid is a salmon or a trout.

Tissues suitable for use in the methods of the invention include, but are not limited to skin, feather pulp, wattle, comb, blood including cellular fractions, egg, epithelium, mucosa, lung, spleen, liver, kidney, conjunctiva, thymus, bursa, fin and gill.

By using a tissue sample comprising, for example, skin and/or feather pulp, the assay may advantageously be performed on live animals. Other suitable tissue samples which may be obtained from live animals include, but are not limited to, wattle, comb, blood, egg, fin and gill.

The present inventors found that influenza virus replicated in tissue explants such as explants of skin and feather pulp. It was previously not known or expected that influenza virus could replicate in these tissue explants.

Accordingly, in one preferred embodiment of the present invention, the tissue sample comprises skin.

Any suitable method for detecting the presence, absence and/or replication of virus in a tissue sample may be employed in the methods of the invention. For example, virus may be detected by detecting viral polypeptides, such as by using specific antibodies, or by detecting viral nucleic acid, by detecting cytopathic effects (CPE) or by any other suitable means known to the person skilled in the art. One example of an assay for detecting Influenza Virus in a sample is the hemagglutination assay.

In one embodiment, detecting the presence or absence of virus in the tissue sample comprises isolating nucleic acid from the tissue sample. The method may further comprise attempting to amplify a viral nucleic acid from the isolated nucleic acid.

In one embodiment, the nucleic acid which is isolated from the tissue sample is RNA.

Viruses that may be detected by the methods of the invention include, but are not limited to, viruses such as Influenza Virus, Newcastle Disease Virus, Infectious Bursal Disease Virus, Foot and Mouth Disease Virus, Porcine Respiratory Reproductive Syndrome Virus, Classical Swine Fever Virus, Bluetongue Virus, Akabane Virus, Infectious Salmon Anemia Virus, Infectious Hematopoietic Necrosis Virus, Viral Haemorrhagic Septicaemia Virus and Infectious Pancreatic Necrosis Virus.

In one embodiment of the invention, the virus is influenza virus.

In an embodiment, the influenza virus is influenza A. The influenza A may be any strain of influenza A, but in one embodiment the influenza A is avian influenza A.

To detect virus or viral replication in a sample, any suitable viral target viral nucleic acid may be amplified. In one embodiment of the invention, the viral nucleic acid that is amplified is a region of the M gene of influenza virus.

In one particular embodiment, the viral nucleic acid comprises at least 15 contiguous nucleotides of SEQ ID NO:1.

In another embodiment, the viral nucleic acid comprises SEQ ID NO:2.

In a preferred embodiment, no cell or tissue culturing is required prior to contacting the tissue sample with the virus.

In performing the method of the present invention, the tissue sample is contacted with the virus so that the virus is able attach to cells in the sample. In one embodiment, the virus is contacted with the tissue sample for between about 15 min and about 2 hours.

In another embodiment, the virus is contacted with the tissue sample for about 1 hour.

The method of the invention further comprises incubating the tissue sample for time sufficient for viral replication. For example, the method may comprise incubating the tissue sample for between about 1 hour and about 48 hours.

In one embodiment, the sample is incubated for about 48 hours.

Typically, the incubation of the tissue sample is performed in an environment that is suitable for maintaining the sample in a viable condition. The skilled person will understand that factors influencing the chosen incubation environment include, for example, the type of tissue sample obtained from the animal, and whether the animal is a warm or cold-blooded vertebrate.

In one embodiment, the incubation is at about 37° C.

In yet another embodiment, the virus is infectious salmon anemia virus.

In one embodiment, the viral nucleic acid that is amplified is a region of Segment 7 or Segment 8 of infectious salmon anemia virus. For example, the nucleic acid may comprise at least 15 contiguous nucleotides of SEQ ID NO:10 or SEQ ID NO:11. In one particular embodiment, the viral nucleic acid comprises SEQ ID NO:12 or SEQ ID NO:13.

In an embodiment, the virus is contacted with the tissue sample for between about 15 min and about 3 hours. In one particular embodiment, the virus is contacted with the tissue sample for about 1.5 hours.

In one embodiment, the method comprises incubating the tissue sample for between about 1 day and about 10 days.

In another embodiment, the method comprises incubating the tissue sample for about 3 days to about 10 days.

In instances where the tissue sample has been obtained from a cold-blooded vertebrate, it may be desirable to incubate the sample at a temperature lower than 37° C. For example, it may be desirable to incubate the tissue sample at about 10° C. to about 20° C. In one particular embodiment, incubation is at about 15° C.

In one particular embodiment, incubation of the tissue sample is performed in a humidified atmosphere containing about 5% CO₂.

The method of the present invention may also be performed on tissue samples obtained from a transgenic animal Such transgenic animals may, for example, comprise a transgene that affects the susceptibility of the animal to a viral pathogen. Accordingly, in one embodiment of the invention, the tissue sample comprises transgenic cells.

In an embodiment, the transgenic cells comprise a transgene encoding a dsRNA molecule, for example, a short-hairpin RNA (shRNA) molecule.

When the transgene encodes a dsRNA molecule, the dsRNA molecule may comprise a viral nucleic acid sequence, or alternatively a nucleic acid sequence that is endogenous to the animal.

In one embodiment, the viral nucleic acid sequence is an influenza A nucleic acid sequence.

In yet another embodiment, the subject has not been exposed to the virus prior to performing the method of the invention.

The present invention further provides a method for identifying an animal having decreased susceptibility to a virus, the method comprising

(i) performing the method of the invention, and

(ii) identifying animals having decreased susceptibility to a virus.

The present invention further provides a method for breeding animals, the method comprising

(i) performing the method of the invention;

(ii) selecting an animal having decreased susceptibility to a virus; and

(iii) breeding from the animal.

In one embodiment, the method for breeding animals further comprises

(i) selecting a first animal of a first gender having decreased susceptibility to the virus; and

(ii) selecting a second animal of the opposite gender having decreased susceptibility to the virus; and

(iii) mating the first and second animals to produce offspring.

The present invention further provides a kit for performing the method of the invention, the kit comprising means for detecting the virus.

In one embodiment, the kit comprises at least one nucleic acid molecule which hybridises under stringent conditions with a viral nucleic acid.

In another embodiment, the at least one nucleic acid molecule is a primer useful for amplifying a viral nucleic acid.

In yet another embodiment, the kit further comprises a sample of virus.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### BRIEF DESCRIPTION OF THE FIGURES

FIG. 1. Influenza A virus replication in explant tissue samples. Skin (A), thumb (also known as “alula”) (B) and feather pulp (C) were taken from 12 day old chicks. The tissue samples were infected with Influenza A PR8 virus and incubated for 1 to 48 hours. RNA was extracted from the infected samples and then assayed by real-time PCR with specific primers for the Influenza A matrix (M) gene. The real time PCR results show a consistent and significant increase in M gene mRNA at 48 hours post infection compared with the 1 hour post infection time point.

FIG. 2. HA results from RCAS transformed CEF cultures expressing shRNA and infected with H5N1 influenza. 1—CEFs integrated with RCAS vector alone. 2—CEFs integrated with RCAS expressing PB1-2257 shRNA.

FIG. 3. Quantitative PCR of Infectious Salmon Anemia Virus in Atlantic salmon explants. The fold increase in ISAV RNA in gill explant samples was determined by qPCR at days 0, 3 and 10 post-infection.

### KEY TO THE SEQUENCE LISTING

SEQ ID NO:1—Coding sequence of the M gene of Influenza A
SEQ ID NO:2—Region of the M gene of Influenza A
SEQ ID NO:3—PB1 sequence targeted by shRNA
SEQ ID NO:4—ISAV S7-F1 oligonucleotide primer.
SEQ ID NO:5—ISAV S7-R1 oligonucleotide primer.
SEQ ID NO:6—ISAV S7-probe.
SEQ ID NO:7—ISAV S8-F1 oligonucleotide primer.
SEQ ID NO:8—ISAV S8-R1 oligonucleotide primer.
SEQ ID NO:9—ISAV S8-probe.
SEQ ID NO:10—Nucleotide sequence of infectious salmon anemia virus Segment 7.
SEQ ID NO:11—Nucleotide sequence of infectious salmon anemia virus Segment 8.
SEQ ID NO:12—Infectious salmon anemia virus Segment 7 amplicon.
SEQ ID NO:13—Infectious salmon anemia virus Segment 8 amplicon.

### DETAILED DESCRIPTION

### General Techniques and Selected Definitions

Unless specifically defined otherwise, all technical and scientific terms used herein shall be taken to have the same meaning as commonly understood by one of ordinary skill in the art (e.g., in cell culture, microbiology especially virology, molecular genetics, immunology, immunohistochemistry, protein chemistry, and biochemistry).

Unless otherwise indicated, the microbiological, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, 3^{rd} ed., Cold Spring Harbour Laboratory Press (2001), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al., (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al., (editors) Current Protocols in Immunology, John Wiley & Sons (including all updates until present).

As used herein, the term “subject” refers to an animal, for example, a bird, fish or mammal and includes a human. In one embodiment, the subject may be avian, for example poultry such as a chicken, turkey or a duck. In other embodiments, the subject may be, e.g., sheep, pig or cattle.

In an embodiment, the subject is a chicken.

In another embodiment, the subject is a salmonid.

The term “avian” as used herein refers to any species, subspecies or race of organism of the taxonomic Class Aves, such as, but not limited to, such organisms as chicken, turkey, duck, goose, quail, pheasants, parrots, finches, hawks, crows and ratites including ostrich, emu and cassowary. The term includes the various known strains of Gallus gallus (chickens), for example, White Leghorn, Brown Leghorn, Barred-Rock, Sussex, New Hampshire, Rhode Island, Australorp, Cornish, Minorca, Amrox, California Gray, Italian Partidge-coloured, as well as strains of turkeys, pheasants, quails, duck, ostriches and other poultry commonly bred in commercial quantities.

The term “poultry” includes all avians kept, harvested, or domesticated for meat or eggs, for example chicken, turkey, ostrich, game hen, squab, guinea fowl, pheasant, quail, duck, goose, and emu.

The term “salmonid” as used herein refers to fish of the Salmonidae family and includes salmon, trout, char and whitefish. Non-limiting examples of salmon include Atlantic salmon, Chinook salmon, pink salmon, coho salmon, cherry salmon, sockeye salmon and chum salmon. Non-limiting examples of trout include rainbow trout, brown trout, brook trout and lake trout.

The “sample” may be of any suitable type and may, by way of non-limiting example, refer to a tissue sample such as skin, feather pulp, wattle, comb, blood including cellular fractions thereof, egg, epithelium, mucosa, lung, spleen, liver, kidney, conjunctiva, thymus, bursa, fins and gills.

As used herein “susceptibility” refers to the ability of an animal to be infected with a virus, including clinical or subclinical infection. By “decreased susceptibility” it is meant a decreased level of susceptibility when compared to a normal population.

By “virus that is not attached to a cell” it is meant a viral particle that does not have a surface protein associated with a specific receptor on the host cell surface.

“Viral replication” as used herein refers to the amplification of the viral genome in a host cell.

As used herein, “avian influenza virus” refers to any influenza A virus that may infect birds. Examples of avian influenza viruses include, but are not limited to, any one or more of subtypes H1 to H16, and N1 to N9, and include highly pathogenic and low pathogenic strains. In one embodiment, the avian influenza virus is of the H5 subtype. In another embodiment, the avian influenza virus is of the H7 subtype. In another embodiment, the avian influenza virus is of the H5N1 subtype.

As used herein, the term “about” refers to a range of +/−5% of the specified value.

### Assays

The methods of the present invention provide assays in which a tissue sample from a subject is contacted with a virus, and after time sufficient for viral replication, the presence or absence of virus in the sample is detected.

In one embodiment, the invention provides a method for detecting viral replication in a tissue sample from an animal, the method comprising:

contacting a tissue sample obtained from the animal with a virus,

incubating the tissue sample for time sufficient for viral replication, and

detecting the presence or absence of virus in the tissue sample.

The skilled person will understand that the conditions under which such an assay is performed may depend on the species from which the tissue sample is derived and/or the virus that is contacted with the tissue sample. For example, factors such as the temperature, humidity, atmospheric composition and time in which a tissue sample is contacted with a virus and subsequently incubated will vary depending on the subject species and species of virus used in the assay. Such conditions could be routinely determined by the skilled person.

For example, in the case of testing for the replication of influenza virus in a chicken skin sample, the sample may be incubated at about 37° C. in a humidified atmosphere containing about 5% CO₂.

The temperature under which the assay is conducted may be at about 37° C., but may be lower or higher depending on the species from which the test sample is obtained, and the species of virus being tested. For example, when testing for replication of virus in a sample obtained from a fish, the sample may be incubated at between at about 8-18° C.

The tissue sample may be contacted with the virus for a suitable time to allow the virus to enter cells in the sample. For example, the tissue sample may be contacted with the virus for between about 15 minutes to about 2 hours. In one embodiment, the virus is contacted with the tissue sample for about 1 hour. In another embodiment, the virus is contacted with the sample for about 1.5 hours.

The tissue sample is then incubated for time sufficient for viral replication. The incubation time may be, for example, about 1 hour to about 48 hours. In instances where the tissue sample is incubated at lower temperatures and/or where viral growth is slower, the incubation time may be about 1 day to about 10 days. The skilled person can readily determine a suitable period of incubation.

The tissue samples to be contacted with virus may be placed in wells of a suitable vessel such as in a microtiter vessel or other multiwell plate. In one embodiment, aliquots (e.g., serially diluted aliquots) of the virus are added to the tissue samples, the virus removed and then the tissue samples incubated under conditions that allow for replication of the virus, which are typically conditions suitable for maintaining the viability of the particular host tissue sample. In one embodiment, following replication of the virus, viral nucleic acid is released by lysis of cells in the tissue sample, using conditions or agents that promote lysis as necessary.

In one embodiment, an attempt is made to amplify a viral nucleic acid from the isolated nucleic acid. The nucleic acid that is amplified may be RNA or DNA.

In other embodiments, nucleic acid, including viral nucleic acid, in a multiplicity of lysates, such as an array, is transferred and fixed to a membrane under conditions that bind nucleic acid (washing as appropriate to remove proteins and other contaminants). Hybridizing the membrane with a labeled virus-specific probe can then be used to identify and quantify the relative amount of viral-specific nucleic acid in each of the points on the array, and by correspondence, in each of the original culture wells. Conditions and materials for nucleic acid transfer, binding, washing and hybridizing can be adapted from routine molecular biological techniques such as “dot blot” hybridization (as described in the art, see, e.g. the molecular biological techniques in Sambrook et al., supra, and Ausubel et al., supra).

Alternatively, the presence or absence of virus in a sample may be determined using protein detection techniques. In one embodiment, antibodies specific for a viral polypeptide are used to detect the presence or absence of virus in the sample. Any suitable means for detecting a viral polypeptide may be used in the methods of the invention.

In the methods of the present invention, it may be desirable to compare the level of viral replication to a control sample or to quantify the level of viral replication in the sample. Such quantification is readily provided by the inclusion of appropriate control samples. An example of an internal control is one or more samples that have a known quantity of virus and/or which are known to not contain virus. Other examples of internal controls may be a tissue sample in which a particular virus of interest is known to replicate, or conversely in which the virus is known not to replicate.

As will be known to those skilled in the art, when internal controls are not included in each assay conducted, the control may be derived from an established data set.

### Detection of Viral Nucleic Acid

By “isolated nucleic acid” we mean a nucleic acid which has generally been separated from the nucleotide sequences with which it is associated or linked in its native state (if it exists in nature at all). Preferably, the isolated nucleic acid is at least 60% free, more preferably at least 75% free, and more preferably at least 90% free from other components with which it is naturally associated.

The terms “nucleic acid molecule” or “polynucleotide” refer to an oligonucleotide, polynucleotide or any fragment thereof. It may be DNA or RNA of genomic or synthetic origin.

In one embodiment, the present invention provides a method for determining the susceptibility of a subject to a virus, the method comprising contacting a tissue sample obtained from the subject with the virus, incubating the tissue sample for time sufficient for viral replication, and detecting the presence or absence of viral replication in the tissue sample, wherein the method further comprises isolating nucleic acid from the tissue sample. In another embodiment, the method further comprises attempting to amplify a viral nucleic acid from the isolated nucleic acid. The skilled person will understand that any suitable technique for detecting a viral nucleic acid may be used in the methods of the present invention.

The skilled person will appreciate that any suitable viral nucleic acid sequence may be detected in the methods of the present invention. Any suitable technique that allows for the detection of a nucleic acid may be used, including those that allow quantitative assessment of the level of expression of a specific gene in a tissue. Comparison may be made by reference to a standard control, or to a control level that is found in uninfected tissue. For example, levels of a transcribed gene can be determined by Northern blotting, and/or RT-PCR. With the advent of quantitative (real-time) PCR, the number of gene copies present in any RNA population can accurately be determined by using appropriate primers for the gene of interest. Levels of a plurality of transcribed genes can be now monitored by hybridisation on gene arrays that contain nucleic acid sequences from all the genes of interest, immobilised on a solid surface. The nucleic acid may be labelled and hybridised on a gene array, in which case the gene concentration will be directly proportional to the intensity of the radioactive or fluorescent signal generated in the array.

### Amplification of Polynucleotides

The “polymerase chain reaction” (“PCR”) is a reaction in which replicate copies are made of a target polynucleotide using a “pair of primers” or “set of primers” consisting of “upstream” and a “downstream” primer, and a catalyst of polymerization, such as a DNA polymerase, and typically a thermally-stable polymerase enzyme. Methods for PCR are known in the art, and are taught, for example, in “PCR” (Ed. M. J. McPherson and S. G Moller (2000) BIOS Scientific Publishers Ltd, Oxford). PCR can be performed on cDNA obtained from reverse transcribing mRNA isolated from biological samples.

A primer is an oligonucleotide, usually of about 15 to about 50 nucleotides in length, that is capable of hybridising in a sequence specific fashion to the target sequence and being extended during the PCR. Amplicons or PCR products or PCR fragments or amplification products are extension products that comprise the primer and the newly synthesized copies of the target sequences. Multiplex PCR systems contain multiple sets of primers that result in simultaneous production of more than one amplicon. Primers may be perfectly matched to the target sequence or they may contain internal mismatched bases that can result in the introduction of restriction enzyme or catalytic nucleic acid recognition/cleavage sites in specific target sequences. Primers may also contain additional sequences and/or modified or labelled nucleotides to facilitate capture or detection of amplicons. Repeated cycles of heat denaturation of the DNA, annealing of primers to their complementary sequences and extension of the annealed primers with polymerase result in exponential amplification of the target sequence. The terms target or target sequence or template refer to nucleic acid sequences which are amplified.

Another nucleic acid amplification technique is reverse transcription polymerase chain reaction (RT-PCR). First, complementary DNA (cDNA) is made from an RNA template, using a reverse transcriptase enzyme, and then PCR is performed on the resultant cDNA.

Another method for amplification is the ligase chain reaction (“LCR”), disclosed in EP 0 320 308. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750 describes a method similar to LCR for binding probe pairs to a target sequence.

Qβ Replicase, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′α-thio-triphosphates in one strand of a restriction site may also be useful in the amplification of nucleic acids in the present invention (Walker et al., 1992a).

Strand Displacement Amplification (SDA) is another method of carrying out isothermal amplification of nucleic acids which involves multiple rounds of strand displacement and synthesis, i.e., nick translation (Walker et al., 1992b).

Target specific sequences can also be detected using a cyclic probe reaction (CPR). In CPR, a probe having 3′ and 5′ sequences of non-specific DNA and a middle sequence of specific RNA is hybridised to DNA that is present in a sample. Upon hybridisation, the reaction is treated with RNase H, and the products of the probe identified as distinctive products that are released after digestion. The original template is annealed to another cycling probe and the reaction is repeated.

Another example of an isothermal amplification technique is LAMP (loop-mediated isothermal amplification of DNA) and is described in Notomi, T. et al., 2000.

Further amplification methods are described in GB Application No. 2 202 328, and in PCT Application No. PCT/US89/01025, and may be used in accordance with the present invention. In the former application, “modified” primers are used in a PCR-like, template- and enzyme-dependent synthesis. The primers may be modified by labelling with a capture moiety (e.g., biotin) and/or a detector moiety (e.g., enzyme). In the latter application, an excess of labelled probes are added to a sample. In the presence of the target sequence, the probe binds and is cleaved catalytically. After cleavage, the target sequence is released intact to be bound by excess probe. Cleavage of the labelled probe signals the presence of the target sequence.

Other nucleic acid amplification procedures include transcription-based amplification systems (TAS), including nucleic acid sequence based amplification (NASBA) and 3SR (Kwoh et al., 1989; WO 88/10315). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerisation, DNA/RNA hybrids are digested with RNase H while double stranded DNA molecules are heat denatured again. In either case the single stranded DNA is made fully double stranded by addition of second target specific primer, followed by polymerisation. The double-stranded DNA molecules are then multiply transcribed by an RNA polymerase such as T7 or SP6. In an isothermal cyclic reaction, the RNAs are reverse transcribed into single stranded DNA, which is then converted to double stranded DNA, and then transcribed once again with an RNA polymerase such as T7 or SP6. The resulting products, whether truncated or complete, indicate target specific sequences.

Methods for direct sequencing of nucleotide sequences are well known to those skilled in the art and can be found for example in Ausubel et al., eds., Short Protocols in Molecular Biology, 3rd ed., Wiley, (1995) and Sambrook et al., Molecular Cloning, 3rd ed., Cold Spring Harbor Laboratory Press, (2001). Sequencing can be carried out by any suitable method, for example, dideoxy sequencing, chemical sequencing or variations thereof. Direct sequencing has the advantage of determining variation in any base pair of a particular sequence.

Hybridization based detection systems include, but are not limited to, the TaqMan assay and molecular beacons. The TaqMan assay (U.S. Pat. No. 5,962,233) uses allele specific (ASO) probes with a donor dye on one end and an acceptor dye on the other end such that the dye pair interact via fluorescence resonance energy transfer (FRET). A target sequence is amplified by PCR modified to include the addition of the labeled ASO probe. The PCR conditions are adjusted so that a single nucleotide difference will effect binding of the probe. Due to the 5′ nuclease activity of the Taq polymerase enzyme, a perfectly complementary probe is cleaved during PCR while a probe with a single mismatched base is not cleaved. Cleavage of the probe dissociates the donor dye from the quenching acceptor dye, greatly increasing the donor fluorescence.

An alternative to the TaqMan assay is the molecular beacon assay (U.S. Pat. No. 5,925,517). In the molecular beacon assay, the probes contain complementary sequences flanking the target specific species so that a hairpin structure is formed. The loop of the hairpin is complimentary to the target sequence while each arm of the hairpin contains either donor or acceptor dyes. When not hybridized to a donor sequence, the hairpin structure brings the donor and acceptor dye close together thereby extinguishing the donor fluorescence. When hybridized to the specific target sequence, however, the donor and acceptor dyes are separated with an increase in fluorescence of up to 900 fold. Molecular beacons can be used in conjunction with amplification of the target sequence by PCR and provide a method for real time detection of the presence of target sequences or can be used after amplification. The skilled person will understand that any suitable method of amplifying or detecting a nucleic acid may be used in the method of the present invention.

### Hybridization

In the methods of the present invention, a viral nucleic acid may be detected by any suitable hybridization technique including, but not limited to, Southern, Northern blot or dot blot analysis. A sample to be tested for the presence or absence of virus may comprise a cell, genomic DNA (such as for Southern blot analysis), RNA (such as for Northern blot analysis), cDNA and the like. If desired, viral or probe nucleic acid may be in solution or immobilised to a solid support such as a microtitre plate, membrane, polystyrene bead, glass slide or other solid phase.

The term “hybridization” as used here refers to the association of two nucleic acid molecules with one another by hydrogen bonding. Factors that affect this bonding include: the type and volume of solvent; reaction temperature; time of hybridization; agitation; agents to block the non-specific attachment of the liquid phase molecule to the solid support (e.g., Denhardt's reagent or BLOTTO); the concentration of the molecules; use of compounds to increase the rate of association of molecules (e.g., dextran sulphate or polyethylene glycol); and the stringency of the washing conditions following hybridization (see Sambrook et al. Molecular Cloning; A Laboratory Manual, Second Edition (1989)).

“Stringency” refers to conditions in a hybridization reaction that favour the association of very similar molecules over association of molecules that differ. High stringency hybridisation conditions are defined as, e.g., overnight incubation at 42° C. in a solution comprising 50% formamide, 5×SSC (150 mM NaCl, 15 mM trisodium citrate, pH8.0), 50 mM sodium phosphate (pH7.6), 5× Denhardts solution, 10% dextran sulphate, and 20 microgram/ml denatured, sheared salmon sperm DNA, followed by washing the filters in 0.1×SSC at approximately 65° C. Low stringency conditions involve the hybridisation reaction being carried out at 35° C. Preferably, the conditions used for hybridization in the methods of the present invention are those of high stringency.

Generally, an oligonucleotide useful as a probe or primer that hybridizes to a viral nucleic acid molecule is at least about 12 to 15 nucleotides in length, or at least about 18 to 20 nucleotides in length, or at least about 21 to 25 nucleotides in length, and optionally about 26 to 35 nucleotides in length or more. Preferably, a nucleic acid molecule hybridizes with a viral nucleic acid molecule at least two times the background and more typically more than 10 to 100 times background.

The nucleic acid is preferably isolated from the sample for testing. Suitable methods will be known to those of skill in the art. For example, RNA may be isolated from the sample to be analysed using conventional procedures, such as are supplied by QIAGEN technology. This RNA is then reverse-transcribed into DNA using reverse transcriptase and the DNA molecule of interest may then be amplified by PCR techniques using specific primers.

### Detection of Viral Polypeptides

In one embodiment, a viral polypeptide or an immunogenic fragment or epitope thereof is detected in a sample, wherein the level of the polypeptide or immunogenic fragment or epitope in the sample is indicative of viral replication. Preferably, the method comprises contacting a protein or immunogenic fragment obtained from the sample with a binding agent capable of binding to a viral polypeptide or an immunogenic fragment or epitope thereof, and detecting the formation of a complex between the binding agent and the viral polypeptide or immunogenic fragment or epitope thereof. In an embodiment, the binding agent is an antibody.

Preferably, a binding agent binds selectively to a viral polypeptide and not generally to other polypeptides unintended for binding. The binding agent is capable of binding a viral polypeptide in the presence of excess quantities of other polypeptides, and tightly enough (i.e. with high enough affinity) that it provides a useful tool for detecting the viral polypeptide. The parameters required to achieve such specificity can be determined routinely, using conventional methods in the art. Preferably, the binding agent binds to a viral polypeptide at least two times the background and more typically 10 to 100 times background.

Detection systems contemplated herein include any known assay for detecting proteins in a biological sample isolated from a subject, such as, for example, SDS/PAGE, isoelectric focussing, 2-dimensional gel electrophoresis comprising SDS/PAGE and isoelectric focussing, an immunoassay, a detection based system using an antibody or non-antibody ligand of the protein, such as, for example, a small molecule (e.g. a chemical compound, agonist, antagonist, allosteric modulator, competitive inhibitor, or non-competitive inhibitor, of the protein). In accordance with these embodiments, the antibody or small molecule may be used in any standard solid phase or solution phase assay format amenable to the detection of proteins. Optical or fluorescent detection, such as, for example, fluorescence-activated cell sorting (FACS), using mass spectrometry, MALDI-TOF, biosensor technology, evanescent fiber optics, or fluorescence resonance energy transfer, is clearly encompassed by the present invention. Assay systems suitable for use in high throughput screening of mass samples, particularly a high throughput spectroscopy resonance method (e.g. MALDI-TOF, electrospray MS or nano-electrospray MS), are also contemplated.

Suitable immunoassay formats include immunoblot, Western blot, dot blot, enzyme linked immunosorbent assay (ELISA), radioimmunoassay (RIA) and enzyme immunoassay. Modified immunoassays utilizing fluorescence resonance energy transfer (FRET), isotope-coded affinity tags (ICAT), matrix-assisted laser desorption/ionization time of flight (MALDI-TOF), electrospray ionization (ESI), biosensor technology, evanescent fiber-optics technology or protein chip technology are also useful.

In one embodiment, the assay is a semi-quantitative assay or quantitative assay.

Standard solid phase ELISA formats are particularly useful in determining the concentration of a viral polypeptide from a variety of samples.

Such ELISA based systems are particularly suitable for quantification of the amount of a viral polypeptide in a sample, such as, for example, by calibrating the detection system against known amounts of a standard.

In another form, an ELISA consists of immobilizing an antibody that specifically binds a viral polypeptide on a solid matrix, such as, for example, a membrane, a polystyrene or polycarbonate microwell, a polystyrene or polycarbonate dipstick or a glass support. A sample is then brought into physical relation with said antibody, and the antigen in the sample is bound or ‘captured’. The bound protein can then be detected using a labelled antibody. For example if the protein is captured from a sample suspected of containing an influenza virus, an antibody against an influenza virus polypeptide is used to detect the captured protein. Alternatively, a third labelled antibody can be used that binds the second (detecting) antibody.

Biosensor devices generally employ an electrode surface in combination with current or impedance measuring elements to be integrated into a device in combination with the assay substrate (such as that described in U.S. Pat. No. 5,567,301). An antibody or ligand that specifically binds to a protein of interest is preferably incorporated onto the surface of a biosensor device and a biological sample isolated from a subject contacted to said device. A change in the detected current or impedance by the biosensor device indicates protein binding to said antibody or ligand. Some forms of biosensors known in the art also rely on surface plasmon resonance to detect protein interactions, whereby a change in the surface plasmon resonance surface of reflection is indicative of a protein binding to a ligand or antibody (U.S. Pat. Nos. 5,485,277 and 5,492,840).

Biosensors are of particular use in high throughput analysis due to the ease of adapting such systems to micro- or nano-scales. Furthermore, such systems are conveniently adapted to incorporate several detection reagents, allowing for multiplexing of diagnostic reagents in a single biosensor unit.

An evanescent biosensor generally relies upon light of a predetermined wavelength interacting with a fluorescent molecule, such as for example, a fluorescent antibody attached near the probe's surface, to emit fluorescence at a different wavelength upon binding of the diagnostic protein to the antibody or ligand.

### Detection of Cytopathic Effects (CPE)

The terms “cytopathic effect” or “CPE” as used herein describe changes in cellular structure (i.e., a pathologic effect). Common cytopathic effects include cell destruction, syncytia (i.e., fused giant cells) formation, cell rounding, vacuole formation, and formation of inclusion bodies. CPE results from actions of a virus on permissive cells that negatively affect the ability of the permissive cellular host to perform its required functions to remain viable.

The presence of a virus often gives rise to morphological changes in the host cell. Any detectable changes in the host cell due to infection are known as a cytopathic effect. Cytopathic effects (CPE) include cell rounding, disorientation, swelling or shrinking, death, detachment from the surface, etc.

In one embodiment of the invention, viral replication in a tissue sample may be detected by determining the presence or absence of cytopathic effects in the sample.

In one particular embodiment, the cytopathic effect is detected by staining the sample with a dye. Suitable dyes for the detection of CPE are know in the art. For example, CPE can be detected by measuring an increase in Neutral Red uptake by cells. A Neutral Red uptake assay may be performed by adding Neutral Red at approximately 0.34% concentration to medium added to a test sample comprising cells. After 2 hours the colour intensity of dye absorbed by cells is determined using, e.g., a microplate autoreader.

### Viral Pathogens

The method of the present invention may be used to determine whether a virus can replicate in a tissue sample from an animal Replication of the virus may indicate susceptibility of the animal to a viral pathogen. Examples of significant viral diseases in poultry include, but are not limited to, avian influenza, Marek's disease, Newcastle disease, infectious bursal disease, infectious anaemia and infectious bronchitis. In pigs, transmissible gastroenteritis, porcine reproductive and respiratory syndrome, classical swine fever, pseudorabies, and rabies infections are serious health problems. An important disease of cloven-hoofed animals is Foot and Mouth Disease (FMD). Significant viral pathogens of fish include Infectious Salmon Anemia Virus, Infectious Hematopoietic Necrosis Virus, Viral Haemorrhagic Septicaemia Virus and Infectious Pancreatic Necrosis Virus. Other examples of viral pathogens known to infect animals include, but are not limited to, bluetongue virus, eubenangee virus, African horse sickness virus, Nebraska calf diarrhea virus, bovine or ovine rotavirus, avian rotavirus, bovine enteroviruses, porcine enteroviruses, Eastern equine encephalitis virus, Venezuelan equine encephalitis virus, Western equine encephalitis virus, Nairobi sheep disease virus, Influenza virus type A, swine influenza virus and equine influenza viruses; distemper virus, Rinderpest virus, bovine respiratory syncytial virus, rabies virus, fish rhabdoviruses, Infectious Bronchitis Virus (IBV), cowpox virus, buffalopox virus, fowlpox virus, sheeppox virus, goatpox virus, swinepox virus, bovine papular stomatitis virus, African swine fever virus, equine abortion virus, equine herpes virus 2 and 3, infectious bovine keratoconjunctivitis virus, infectious bovine rhinotracheitis virus, adenoviruses of cattle, pigs, sheep and many other species, avian adenoviruses, bovine papilloma viruses, bovine parvovirus, and Akabane virus.

### Influenza Virus

An example of an important viral pathogen is the influenza virus. Three types of influenza viruses, types A, B, and C are known and they belong to a family of single-stranded negative-sense enveloped RNA viruses called Orthomyxoviridae. The viral genome is approximately 12,000 to 15,000 nucleotides in length and comprises eight RNA segments (seven in Type C) that encode eleven proteins.

Influenza A virus infects many animals such as humans, pigs, horses, marine mammals, and birds and infects epithelial cells of the respiratory tract. Its natural reservoir is in aquatic birds, and in avian species most influenza virus infections cause mild localized infections of the respiratory and intestinal tract. However, the virus can have a highly pathogenic effect in poultry, with sudden outbreaks causing high mortality rates in affected poultry populations.

Influenza A viruses can be classified into subtypes based on allelic variations in antigenic regions of two genes that encode surface glycoproteins, namely, hemagglutinin (HA) and neuraminidase (NA) which are required for viral attachment and cellular release. Other major viral proteins include the nucleoprotein, the nucleocapsid structural protein, matrix proteins (M1 and M2), polymerases (PA, PB1 and PB2), and non-structural proteins (NS1 and NS2).

At least sixteen subtypes of HA (H1 to H16) and nine NA (N1 to N9) antigenic variants are known in influenza A virus. Avian influenza strains can also be characterized as low pathogenic and highly pathogenic strains. Low pathogenic strains typically only have two basic amino acids at positions-1 and -3 of the cleavage site of the HA precursor, while highly pathogenic strains have a multi-basic cleavage site. Subtypes H5 and H7 can cause highly pathogenic infections in poultry and certain subtypes have been shown to cross the species barrier to humans. Highly pathogenic H5 and H7 viruses can also emerge from low pathogenic precursors in domestic poultry. Symptoms of avian influenza infection range from typical influenza type symptoms (fever, cough, sore throat and muscle aches) to conjunctivitis, pneumonia, acute respiratory distress, and other life-threatening complications.

The methods of the present invention can be used to identify animals that have a decreased susceptibility to influenza virus infection. Any suitable method for detecting influenza virus may be used in the methods of the present invention. When detecting influenza nucleic acid, the nucleic acid sequence to be detected may be any of the influenza genes or a region thereof, i.e., the genes encoding the M1 matrix protein, M2 matrix protein, neraminidase (NA), hemagglutinin (HA), non-structural protein 1 and 2 (NS1 and NS2), nucleocapsid protein (NP), polymerase (PA), polymerase 1 (PB1) or polymerase 2 (PB2). In one embodiment, the nucleic acid sequence that is amplified is a region of the M gene of influenza virus. In an embodiment, the viral nucleic acid comprises at least 15 nucleotides of SEQ ID NO:1, and may, for example, comprise SEQ ID NO:2. Alternatively, the polypeptides encoded by any of the influenza virus genes may be detected.

### Newcastle Disease Virus

Newcastle disease (ND) is a serious illness of poultry which is often times fatal, and therefore can result in significant economic losses. The ailment is caused by the Newcastle disease virus (NDV), a virus belonging to the genus Paramyxovirus of the family Paramyxoviridae.

The Newcastle disease virus enters the animal's body via the respiratory and intestinal tract. Symptoms of Newcastle disease are primarily respiratory and nervous. Gasping is common Nervous symptoms include unilateral and bilateral paralysis of wings and/or legs, circular movements, bobbing/waving movements of the head and neck, and spasms of the wing, neck or leg muscles. General symptoms can include loss of appetite and decreased egg laying, often by as much as 40% or more.

Mortality can vary, depending on the properties of the virus involved and the immune status of the particular flock. Generally, those strains that kill quickly spread less between affected birds than those killing more slowly. In addition, a long asymptomatic carrier state has been presumed to occur in certain species of poultry such as chickens. The greatest risk of spreading the disease during an outbreak comes form movement of people and equipment. Due to centralization of many processes in the poultry industry, there is considerable traffic of personnel and equipment moving from one flock to another.

Unfortunately, at present there appears to be no way to distinguish vaccinated members of a flock from those unvaccinated members that have-been afflicted with the virulent, “wild-type” version of Newcastle disease virus. In both instances, antibodies are produced in the animals' bodies. However, current vaccination immunogens against NDV induce antibodies that are very often indistinguishable from antibodies found after infection with virulent, infectious NDV.

The methods of the present invention allow for the identification of poultry with a decreased susceptibility to NDV. The NDV nucleic acid sequence which is detected may be from any genomic gene sequence or a region thereof, for example, such as from the gene encoding the nucleocapsid protein (NP), phosphoprotein (P), matrix protein (M), fusion protein (F), hemagglutinin-neuraminidase (HN) or large polymerase protein (L). Alternatively, a Newcastle Disease Virus polypeptide may be detected in the methods of the invention.

### Chicken Anaemia Virus

The CAV virus causes infectious anaemia in chickens. The virus was first isolated in Japan in 1979 and was given its name because of the serious anaemia caused by it in young chicks (Yuasa, et al., 1979). The other symptoms of CAV infection are the atrophy of the bone marrow and destruction of lymphocytes in the thymus. Lesions occur in the spleen and liver.

Day-old chicks are most susceptible. In these animals lethargy, anorexia and a passing anaemia are observed from four to seven days after inoculation with CAV and about half of the animals die between two and three weeks after infection. With increasing age, the natural resistance also increases. Upon infection at the age of seven days, the chicks only develop a passing anaemia after infection, and upon infection of 14-day-old animals, no anaemia follows.

CAV seems to be spread all over the world. A considerable time after the CAV research had started in Japan, the first CAV isolations were conducted in Europe, namely, in Germany by Von Bulow (1983) and later by McNulty et al. (1990) in the United Kingdom. The available literature data indicate that the isolates belong to one serotype but several field isolates are to be tested for their mutual relationship and possible differences in pathogenicity (McNulty et al., 1990). The spread of CAV within a flock probably occurs by infection via faeces and air. Vertical transmission of virus to the offspring also plays an important role in CAV epidemiology.

When detecting chicken anaemia virus (CAV) using the methods of the invention, the nucleic acid sequence which is detected may be from any CAV gene, for example, CAVgp1, CAVgp2 or Cux-1, or alternatively a polypeptide encoded by a CAV gene is detected.

### Infectious Bursal Disease Virus

Infectious bursal disease (IBD) is an acute contagious viral disease of young chickens also known as Gumboro disease (Kibenge et al., 1988; Lasher et al., 1997). The etiological agent, IBD virus (IBDV), has a predilection for the cells of the bursa of Fabricius where the virus infects actively dividing and differentiating lymphocytes of the B-cell lineage (Burkhardt and Muller, 1987). IBD is a fatal immunosuppressive disease causing heavy losses to the poultry industry.

The first outbreak of IBDV was reported in commercial chicken flocks in Delaware, USA (Cosgrove, 1962). The IBDV strains, which were isolated during the outbreak, now referred to as classical serotype I isolates. The disease was also first report in Europe in 1962. And from 1966 to 1974, IBD was reported in the Middle East, Southern and Western Africa, India, the Far East and Australia. In most cases, the IBDV strains that associated with the outbreaks were of low virulence and caused only 1 to 2% of specific mortality (van den Berg et al., 2000).

In the 1990s, IBDV isolates, which were able to break through levels of maternal antibodies that normally were protective, were reported in Europe. These isolates, the so called very virulent IBDV are causing more severe clinical signs during an outbreak which mortality approaching 100% in susceptible flocks, and are now found almost world-wide (van den Berg et al., 2000). The emergence of very virulent strains of IBDV (vvIBDV) has complicated the immunization programs against the disease.

Early vaccination may result in failure due to interference with the maternal antibody, whilst its delay may cause field virus infections. The identification and of chickens with decreased susceptibility to IBDV infection will allow for breeding and selection of disease resistant lines.

In one embodiment of the invention, Infectious Bursal Disease Virus nucleic acid is detected. The nucleic acid may be from the IBDVsAgp1, IBDVsAgp2 or IBDVsBgp1. Alternatively, a polypeptide encoded by an IBDV gene is detected.

### Foot and Mouth Disease Virus

Foot and mouth disease (FMD) is one of the most serious livestock diseases caused by an apthovirus. It is found in most parts of the world with at least 52 countries throughout Africa, the Middle East, Asia and South America that have reported the disease. There are seven serotypes of the virus: A, O, C, SAT1, SAT2, SAT3 and Asia1. These are further subdivided into more than 60 strains. FMD affects cloven-hoofed animals (those with divided hoofs), including cattle, buffalo, camels, sheep, goats, deer and pigs.

FMD is spreads rapidly between animals in breath, saliva, mucus, milk or faeces. The disease spreads most commonly through the movement of infected animals. It can also be spread on wool, hair, grass or straw, by the wind or by mud or manure sticking to footwear, clothing, livestock equipment or vehicle tyres.

Although FMD is not very lethal in adult animals, it can kill young animals and cause serious production losses. The clinical signs are fever followed by the appearance of vesicles (fluid-filled blisters) between the toes and on the heels, on mammary glands and especially on the lips, tongue and palate. These vesicles often combine to form large, swollen blisters that erupt to leave raw, painful ulcers that take up to 10 days to heal. Foot lesions leave animals lame and unable to walk to feed or water. Tongue and mouth lesions are very painful and cause animals to drool and stop eating. Adults usually begin eating again after a few days, but young animals may weaken and die, or be left with foot deformities or damage to the mammary glands.

FMD is important in international trade in animals and animal products, with countries that are free of the disease banning or restricting imports from affected countries. This means an outbreak would have serious economic implications for any major livestock-exporting country.

The FMD virus contains a positive-strand RNA genome of approximately 8500 nucleotides which is composed of a 5′ untranslated region, the coding region and a 3′ untranslated region. The genome encodes a single polyprotein from which the different viral polypeptides are cleaved. Thus, the methods of the invention may comprise detecting a FMD virus genome or product thereof, for example the structural proteins VP1, VP2, VP3 and VP4, or the non-structural proteins L^{pro}, 3D^{pol}, 2A, 2B, 2C, 3A, 3B, 3C^{pro}, or 3D^{pol}.

### Porcine Reproductive and Respiratory Syndrome Virus

PRRS is a viral disease of pigs, characterized by reproductive failure in sows (e.g., late-term abortions and stillbirths in sows) and respiratory difficulties in piglets (e.g., interstitial pneumonia in nursery pigs) (Collins et al., 1992; and Wensvoort et al., 1991). It was detected in North America in 1987 and in Europe in 1990.

The causative agent is a small, enveloped positive-stranded RNA virus that is recovered primarily from alveolar macrophages and blood of infected swine. It is a member of the Arteriviridae, which includes equine arteritis virus (EAV), lactate dehydrogenase elevating virus of mice (LDV) and simian hemorrhagic fever virus (SHFV). Like other arteriviruses, PRRS virus infects predominantly macrophages and establishes a persistent infection in resident macrophages of numerous tissues (Lawson et al., 1997; and Christopher-Hennings et al., 1995).

For arteriviruses, such as PRRSV, the host susceptibility factors have not been studied. Thus, the markers for pig breeding for host susceptibility to PRRSV are not known. However, it is known that different breeds of pigs do differ in PRRSV susceptibility based on experimental infection followed by sacrificing the animals followed by further examination with histopathology and immunohistochemistry for interstitial pneumonia and presence of PRRSV antigen in the lungs.

Accordingly, the present invention provides a method which could be used to screen pigs for susceptibility to PRRSV infection. Additionally, the screening results could be used in a breeding program designed to lessen the susceptibility of offspring to PRRSV infection.

Porcine Reproductive and Respiratory Syndrome Virus nucleic acid which may be detected in the methods of the present invention may be from the PRRSVgp1, PRRSVgp2, PRRSVgp3, PRRSVgp4, PRRSVgp5, PRRSVgp6, PRRSVgp7, or PRRSVgp8 gene. In another embodiment, the polypeptide encoded by a PRRSV gene is detected.

### Classical Swine Fever Virus

Classical swine fever (CSF), also known as hog cholera or swine fever, is a highly contagious viral disease of pigs. CSF spreads rapidly via contaminated faeces, urine, nasal secretions and tears. Direct contact of infected pigs with susceptible pigs is the most important means of spread, but the virus can also be transmitted on contaminated pens, pig crates, trucks or clothing. Swill-feeding of pigs with infected meat scraps is also an important means of spreading CSF to new areas or countries.

Acute CSF causes sudden fever. Affected pigs first appear drowsy but are later severely depressed and off their feed. They huddle together, stagger and occasionally have convulsions and trembling; vomiting, coughing and diarrhoea are common There is often also red or purple blotching on the skin of the ears, snout, limbs and abdomen of infected animals. Mortalities can reach 90 percent. The chronic form of the disease produces similar clinical signs, though in milder form; death usually results after 30 days or more and is often associated with secondary bacterial infections.

The genome of classical swine fever virus (CSFV) is a single strand RNA of positive sense that is approximately 12,300 nucleotides in length. It has a non-translated region at either end (5′NTR and 3′NTR) and a single open reading frame encoding a large protein (PestiV2gp1 polyprotein) that is cleaved into smaller fragments. Thus, in the methods of the present invention the presence of CSFV may be determined by detecting the PestiV2gp1 gene or gene products, for example by detecting CSFV polyprotein, N-Pro, capsid protein, RNAse, envelope glycoproteins E1 and E2, E2*, non-structural protein p7, NTPase/RNA helicase, non-structural proteins NS4A, NS4B and NS5A or the RNA-dependent RNA polymerase.

### Bluetongue Virus

Bluetongue (BT) is an arthropod-borne infectious viral disease of ruminants. Cattle and goats may be readily infected with the causative BTV but without extensive vascular injury and therefore these species generally fail to show pronounced clinical signs. In contrast, the disease in sheep is characterized by catarrhal inflammation of the mucous membranes of the mouth, nose and forestomachs, and by inflammation of the coronary bands and laminae of the hoofs. There is an excoriation of the epithelium, and ultimately necrosis of the buccal mucosa; the swollen and inflamed tongue and mouth can take on a blue color from which the disease is named (Spreull, 1905). The mortality rate in sheep is estimated at 1-30%.

BTV is the prototype virus of the Orbivirus genus (Reoviridae family) and is made up of at least 24 different serotypes (Wilson et al., 2000). Different strains of BTV have been identified world-wide throughout tropical and temperate zones. BTV is not contagious between ruminants thus the distribution of BTV is dependent on the presence of arthropod vector species of coides sp. (biting midges), with different vector species occurring in different regions of the world. Recent data suggests that genetic drift and founder effect contribute to diversification of individual gene segments of field strains of BTV (Bonneau et al., 2001). It has been shown that BTV seropositive animals are resistant to reinfection with the homologous BTV serotype.

To determine the presence or absence of Bluetongue virus, a Bluetongue virus gene or polypeptide encoded by a Bluetongue virus gene may be detected. Bluetongue virus genes include the BTVs1gp1, BTVs2gp1, BTVs3gp1, BTVs4gp1, BTVs5gp1, BTVs6gp1, BTVs7gp1, BTVs8gp1, BTVs9gp1 and BTVs10gp1 gene.

### Akabane Virus

Akabane is an insect-transmitted virus that causes congenital abnormalities of the central nervous system in ruminants. Disease due to Akabane virus has been recognized in Australia, Israel, Japan, and Korea; antibodies to it have been found in a number of countries in southeast Asia, the Middle East, and Africa. The disease affects fetuses of cattle, sheep, and goats. Asymptomatic infection has been demonstrated serologically in horses, buffalo, and deer (but not in humans or pigs) in endemic areas.

The incidence of Akabane virus-induced disease is influenced by the time of gestation at which infection occurs and also by the strain of virus. Infections in the last 3 months of pregnancy result in a relatively low incidence of disease (5-10% of calves are affected). The peak incidence is seen after infection in the third and fourth months, when up to 40% of calves may be born with defects. Some strains of Akabane virus produce a very low incidence of abnormalities (<20%), even at the most susceptible stages of gestation, whereas the most severe can cause disease in up to 80% of infected animals.

In the methods of the present invention, any genomic Akabane nucleic acid or polypeptide may be detected. Akabane virus genes include the AKAV sSgp1 (nucleocapsid), AKAV sSgp2 (nonstructural protein), AKAV sMgp1 (M gene) and AKAV sLgp1 (Pol) genes.

### Infectious Salmon Anemia Virus

Infectious salmon anemia (ISA) has caused considerable economic losses in the Atlantic salmon farming industry in Norway, Atlantic Canada, and Scotland. Mortality from ISA disease is variable, ranging from 10% to more than 50%. Clinical signs of the disease are apparent in Atlantic salmon, but other salmonids can act as non-symptomatic reservoirs for the virus. The pathological changes associated with ISA are characterized by severe anemia, leukopenia, ascites and hemorrhaging of internal organs with subsequent necrosis of hepatocytes and renal interstitial cells. The infectious agent is an enveloped virus (ISAV) which replicates in endothelial cells in vivo and buds from the cell surface. The virus has a linear, single-stranded negative sense RNA genome consisting of 8 segments ranging in length from approximately 1.0 to 2.2 kb, with a total size of approximately 14.3 kb. The structural, morphological, and physiochemical properties of the virus suggest that ISAV is related to members of the Orthomyxoviridae family (see, e.g., Falk et al., 1997).

The elimination of ISA disease through the attempted eradication of ISA virus has proven to be ineffective. Given the many unknown factors involved in disease transmission, including the reservoirs of virus in wild fish, outright elimination of ISA and the virus (ISAV) does not appear to be an achievable goal.

In the methods of the present invention, any ISAV nucleic acid or polypeptide may be detected. For example one or more of the PB2 polymerase, PB1, NP, P2, P3, HA, P4, P5, P6 or P7 genes or gene products may be detected in the methods of the invention. An assay to detect nucleic acid from Segment 7 (SEQ ID NO:10) or Segment 8 of ISAV is described by Plarre et al. (2005). The primers S7-F1 (SEQ ID NO:4) and S7-R1 (SEQ ID NO:5) may be used to amplify an 81 by region of Segment 7 (SEQ ID NO:12), which can then be detected with the fluorescently-labelled probe 57-P1 (SEQ ID NO:6). The primers S8-F1 (SEQ ID NO:7) and S8-R1 (SEQ ID NO:P8) may be used to amplify a 63 by region of Segment 8 (SEQ ID NO:13) which may be detected with the fluorescently-labelled probe S8-P 1 (SEQ ID NO:9).

### Infectious Hematopoietic Necrosis Virus

Infectious Hematopoietic Necrosis Virus (IHNV) is a rhabdovirus that causes disease in salmonids, such as salmon and trout species. Species that may be infected by IHNV include rainbow/steelhead trout (Oncorhynchus mykiss), cutthroat trout (Salmo clarki), brown trout (Salmo trutta), Atlantic salmon (Salmo salar), Pacific salmon including chinook (O. tshawytscha), sockeye/kokanee (O. nerka), chum (O. keta), masou/yamame (O. masou), amago (O. rhodurus) and coho (O. kisutch).

The IHNV virus is enzootic in the Pacific Northwest portion of the United States as outbreaks of the disease have been reported in Washington, Oregon, and California. The virus has spread beyond the Pacific Northwest and has been reported in other states of the United States, such as Minnesota, Montana, South Dakota, Alaska, and West Virginia, and in Canadian provinces, including British Columbia. The range of the virus now appears to be worldwide as outbreaks have occurred in France, Italy, Belgium, Japan, Taiwan, and Korea.

IHNV infections typically cause severe mortality in young fish, fry, or fingerlings, with reports of up to 80% mortality or severe deformity. Infected fish exhibit externally visible signs of the disease within a week of exposure. Death occurs within four to ten days following exposure, but typically deaths from IHNV cease after about 40 to 50 days.

IHNV, like other rhabdoviruses, is a negative sense RNA virus, the genome of which encodes six genes. The reservoirs of IHNV are clinically infected fish and inapparent carriers among fish. The transmission of IHNV between fish is primarily horizontal, with virus being shed via feces, urine, sexual fluids and external mucus. Cases of vertical transmission, through infected eggs, have also been observed. Once IHNV is established in a population of susceptible fish, the disease is difficult to eliminate because it may become established among carrier fish.

Vaccines are currently under development and testing. However, no vaccine has yet been found to control IHNV infection. Therefore, present control measures for the disease require the identification of infected individuals and measures to prevent uninfected fish from coming into contact with infected individuals and infected environments.

For Infectious Hematopoietic Necrosis Virus, the nucleic acid sequence which may be detected in the methods of the invention include the IHNVgp1 (nucleocapsid), IHNVgp2 (polymerase-associated protein), IHNVgp3 (matrix protein), IHNVgp4 (glycoprotein), IHNVgp5 (non-virion protein) and IHNVgp6 (RNA polymerase) genes. In an alternative embodiment, a polypeptide encoded by an IHNV gene is detected.

### Viral Haemorrhagic Septicaemia Virus

Among the rhabdoviruses that affect fish (novirhabdoviruses), viral haemorrhagic septicaemia virus (VHSV) is one of the most dangerous, as it not only affects salmonids but also cod, turbot, croaker, eels, John Dory and prawns. Despite many efforts a commercial vaccine against VHSV is not yet available.

The infection caused by rhabdovirus begins when the virus binds, by means of the pG glycoprotein, to specific receptors in the outer membrane of the host, followed by membrane fusion dependent on a reduction in pH after the virus has entered the cytoplasm of the cell by endocytosis. Once inside the cells, the rhabdovirus replicates in the cytoplasm, the virions mature and, finally, they bud from the cell surface, lysing the cell.

Due to the significant incidence of VHSV infections, and the lack of available commercial vaccines, it would be desirable to be able to identify fish that have a decreased susceptibility to infection by VHSV.

The nucleic acid sequences which may be detected in the methods of the invention include the genes encoding the N protein (nucleoprotein; VHSVgp1), P protein (phosphorylated protein; VHSVgp2), M protein (matrix protein; VHSVgp3), G protein (glycoprotein; VHSVgp4); NV protein (non-virion protein; VHSVgp5) and L protein (large protein; VHSVgp6). Alternatively, a polypeptide encoded by a VHSV gene is detected.

### Infectious Pancreatic Necrosis Virus

Infectious pancreatic necrosis virus (IPNV) is a member of the family Birnaviridae and causes contagious viral disease of aquatic animals. In fish, IPNV causes morbidity and mortality in rainbow trout, Atlantic salmon, Pacific salmon, brook trout and other salmonids, especially fry, smolt and juvenile stages. IPNV has also been isolated in a variety of aquatic animal species such as carp, perch, pike, eels, char, molluscs and crustaceans.

After an IPNV outbreak, the surviving fish generally become carriers of the virus. The persistence of the virus in carrier fish appears to be due to continuous viral production by a small number of infected cells in certain organs. The only control method currently available for eliminating the virus in carrier fish is to destroy the fish.

The IPNV nucleic acid sequences which may be detected in the methods of the invention include the genes encoding the hypothetical protein (IPNVsAgp1), polyprotein (IPNVsAgp1) and viral protein 1 (IPNVsBgp2). Alternatively, a polypeptide encoded by an IPNV gene is detected.

### Kits

The invention also relates to kits that are useful for detecting viral replication. Such kits may be suitable for detection of nucleic acid species, or alternatively may be for detection of a gene product.

For detection of nucleic acid, such kits may contain a first container such as a vial or plastic tube or a microtiter plate that contains an oligonucleotide probe. The kits may optionally contain a second container that holds primers. The probe may be hybridisable to viral DNA and the primers are useful for amplifying this DNA. Kits that contain an oligonucleotide probe immobilised on a solid support could also be developed, for example, using arrays (see supplement of issue 21(1) Nature Genetics, 1999).

For PCR amplification of nucleic acid, nucleic acid primers may be included in the kit that are complementary to at least a portion of a gene that encodes a viral protein. The set of primers typically includes at least two oligonucleotides that are capable of specific amplification of DNA. Fluorescent-labelled oligonucleotides that will allow quantitative PCR determination may be included (e.g. TaqMan chemistry, Molecular Beacons). Suitable enzymes for amplification of the DNA, may also be included.

Control nucleic acid may be included for purposes of comparison or validation. Such controls could either be RNA or DNA isolated from a tissue sample that has not been incubated with virus, or which is known to be free of virus.

For detection of proteins, antibodies will most typically be used as components of kits. However, any agent capable of binding specifically to a viral polypeptide of interest will be useful in this aspect of the invention. Other components of the kits will typically include labels, secondary antibodies, substrates (if the gene is an enzyme), inhibitors, co-factors and control gene product preparations to allow the user to quantitate expression levels and/or to assess whether the diagnosis experiment has worked correctly. Enzyme-linked immunosorbent assay-based (ELISA) tests and competitive ELISA tests are particularly suitable assays that can be carried out easily by the skilled person using kit components.

### Breeding

Breeding programs for livestock animals typically are designed to breed advantageous characteristics, e.g., disease resistance, into commercial lines. The methods of the present invention can therefore be used advantageously to identify and select animals with resistance, or decreased susceptibility, to particular diseases.

In one embodiment, the present invention provides a method for identifying an animal having decreased susceptibility to a virus, the method comprising

(i) performing the method of the invention, and

(ii) identifying animals having decreased susceptibility to a virus.

Once an animal with decreased susceptibility to a virus has been identified, it can be crossed with an animal of the opposite gender, which may or may not also have a decreased susceptibility to the virus. The resultant progeny may have a decreased susceptibility to the virus similar to the parental animal which has a decreased susceptibility to the virus, or the progeny may have a level of susceptibility intermediate to the parent animals' level of susceptibility to the virus.

Thus, the present invention provides a method for breeding animals, the method comprising

(i) performing the method of the invention;

(ii) selecting an animal having decreased susceptibility to a virus; and

(iii) breeding from the animal.

In one embodiment, the method comprises

(i) selecting a first animal of a first gender having decreased susceptibility to a virus; and

(ii) selecting a second animal of the opposite gender having decreased susceptibility to the virus; and

(iii) mating the first and second animals to produce offspring.

### EXAMPLES

### Example 1

### Preparation of Tissue Samples

Chicken skin (from around breast area beneath wing), thumb (alula) and feather follicle was taken from 12 day old chicks & put into PBSA supplemented with penicillin (100 U/ml) and streptomycin (100 μg/ml) at room temperature. Samples were received & processed within 1.5 hours of being taken from the birds for infection with the PR8 strain of influenza A virus. Skin samples were cut up into approximately 2 mm pieces and feather follicles were cut into 1 cm pieces and the pulp squeezed out of them, the pulp from these follicles being a similar size to the skin tissue pieces. Individual tissue pieces were placed into a 96 well plate containing 200 μl of PBSA.

### Example 2

### Viral Assay

Influenza A PR8 stock virus (stock of allantoic fluid from ten day old embryonated chicken eggs) was serially diluted 10 fold to 10⁻⁵ (1000 TCID50 infectious doses) in Viral Growth Medium (VGM), Earls Modified Eagle's Medium containing 0.3% Bovine Serum Albumin (BSA), 10 mM Hepes, 2 mM glutamine, supplemented with penicillin (100 U/ml), streptomycin (100 μg/ml), fungizone (0.005 μg/ml) and 5 μg/ml trypsin. PBSA was removed and 200 μl of virus added to each well, cultures were incubated at 37° C. for one hour in a humidified atmosphere containing 5% CO₂, the virus was then removed and 200 μl of VGM with 5 μg/ml trypsin was added, the cultures were then incubated at 37° C. in a humidified atmosphere containing 5% CO₂ for either 1 hour or 48 hours.

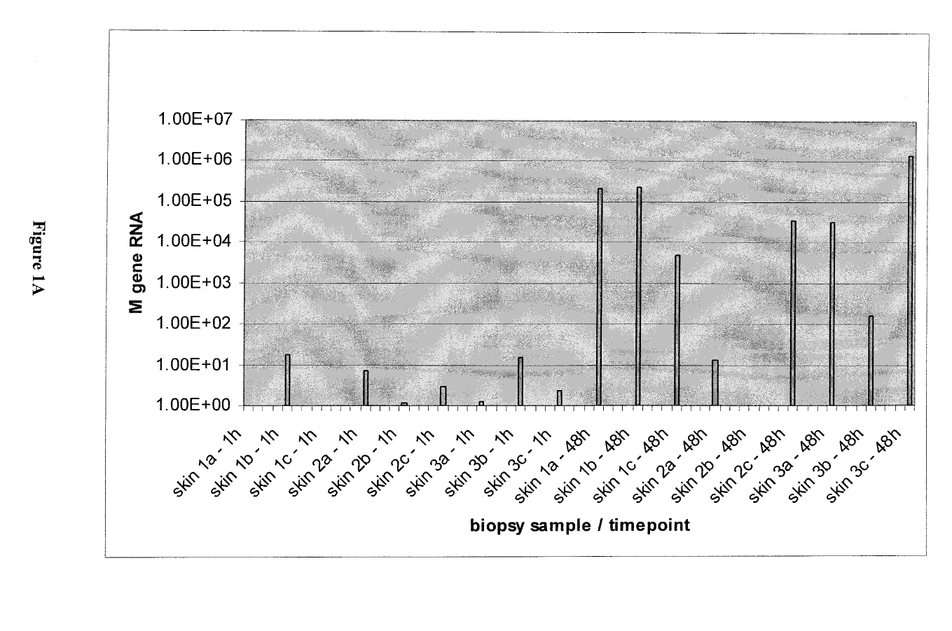

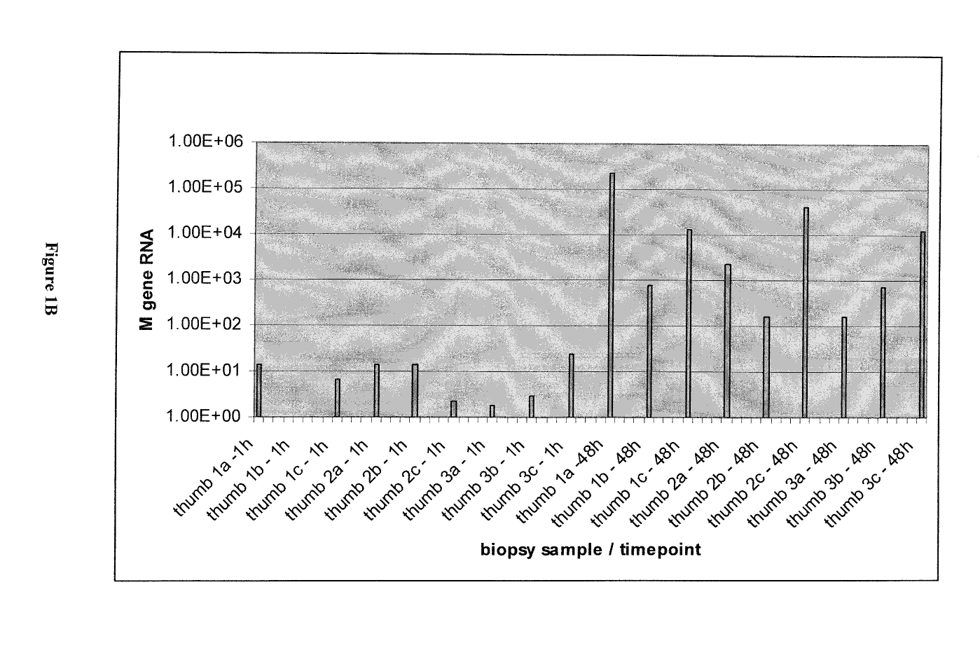

Viral supernatants were removed and tissues washed in 200 μl of PBSA, then processed according to Qiagen RNeasy kit instructions for RNA isolation after spinning through QIAshredder column. RNA was assayed by real-time PCR with specific primers against Influenza A M gene (Heine et al., 2007). Samples were analysed on an ABI 7700 sequence detection system (Applied Biosystems). All experimental conditions were repeated in triplicate and then assayed in triplicate by real-time PCR. The real-time PCR results showed a consistent and significant increase in Influenza A M gene mRNA of up to 5 logs at 48 hours post infection compared with the 1 hour post infection time point (see FIG. 1). This indicates that the virus is successfully replicating in the different cell types.

### Example 3

### Determining Viral Susceptibility in a Chicken Embryo Model

Day 4 specific pathogen free (SPF) embryos were injected with RCAS virus expressing EGFP & a shRNA which targeted the PB1 gene of Influenza A virus (PB1-2257-5′gatctgttccaccattgaa 3′ (SEQ ID NO:3). A small hole was made in the blunt end of the egg exposing the air sac membrane, embryo and blood system. A few microlitres of blood was removed from a vein using a microcaplliary, and 1-2 μl of each RCAS virus (titre approx 10⁸/ml) was then injected via another vein. The egg was then double sealed using 2 small pieces of parafilm which were placed over the opening and sealed using a mildly heated scalpel blade to attach the parafilm to the eggshell surface. The eggs were then incubated until day 9.

Embryos were removed from the egg and screened for EGFP under a dissecting microscope with fluorescent capabilities. The embryos containing the most extensive EGFP expression were selected and Chicken Embryonic Fibroblasts (CEFs) were harvested from these embryos. CEFs were produced by removing the head, limbs and viscera, mincing the remaining parts of the embryo and treating in 0.25% trypsin for 30 minutes at 37° C. with constant stirring. Larger tissue clumps were then filtered out by passing through a 70 μm filter and the remaining cells were then pelleted and resuspended in growth media prior to seeding into tissue culture flasks. The CEFs were trypsinised from flasks after becoming confluent and re-seeded into 24 well plates and allowed to grow to confluency over a few days.

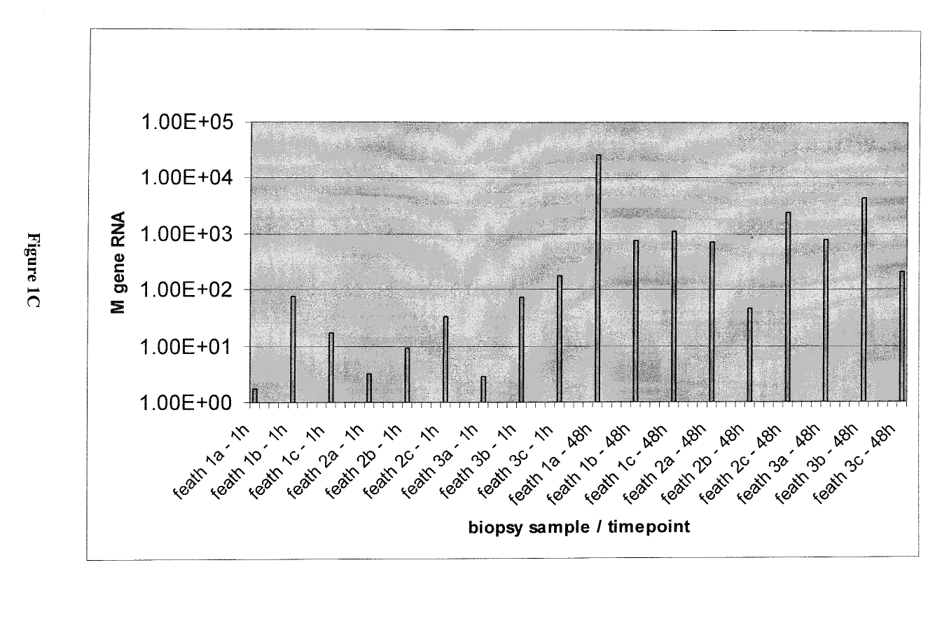

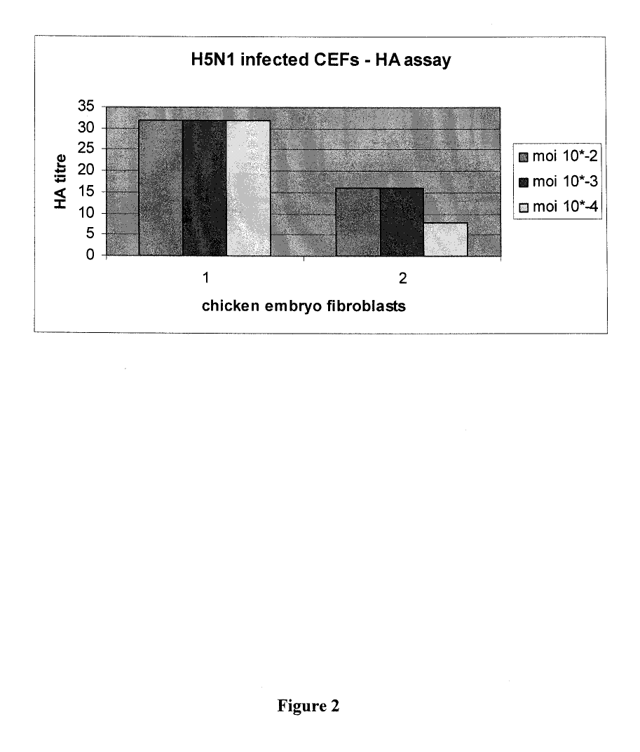

These cultures were checked to ensure they had EGFP expression under a fluorescence microscope prior to infection with Highly Pathogenic Avian Influenza (HPAI)-H5N1 at multiplicities of infection of 0.01, 0.001 and 0.001. The cells were incubated with the virus for one hour at 37° C., the virus removed and fresh media added. The cultures were returned to 37° C. and subsequently incubated for 48 hours. Supernatant from each well was removed and assessed for viral load by haemagglutination assay. 50 μl of viral supernatant was serially diluted 2 fold in PBSA, and 50 μl of prewashed chicken red blood cells at a packed cell volume of 0.5% was added to each well and incubated at room temperature for 45 minutes. Agglutination of red blood cells indicates the presence of virus. CEFs infected with RCAS virus expressing shRNA PB1-2257 were compared to control CEFs infected with RCAS virus lacking the hairpin for their ability to replicate HPAI-H5N1. The CEFs containing the PB1-2257 had increased levels of resistance compared with the control CEF cells (FIG. 2).

### Example 4

### Viral Assay in Atlantic Salmon Explants

Two Atlantic salmon were anaesthetised and blood samples taken from the tail. Blood was collected into Alsevers solution (4 ml of blood into 10 ml of Alsevers). Blood was washed 4 times in wash medium: PBS-ABC with antibiotics (1× gentamycin, 2× Penicillin and streptomycin and 1× fungizone). The cells were pelleted between washes by centrifugation at 500 g for 10 minutes. The buffy coat and a trace of red blood cells, in 3.3 ml, were then used in the experiment as described below.

Once the fish had been bled-out the gills were taken from one fish and placed into wash medium (described above) and held at room temperature for one hour. The medium was then removed. Fresh medium was added and the tubes were allowed to stand for 30 minutes at room temperature. This step was repeated. Finally the last wash was removed and replaced with 1 ml of SHK-1 growth medium.

Six 2 ml Sarstedt tubes were dosed with 1 ml of SHK-1 growth medium. Three of these were dosed with 100 μl of buffy coat. Into each of the other three tubes, a segment of gill was inserted. One of each of gill or buffy coat tubes were dosed with one of three dose of Infectious Salmon Anaemia Virus (ISAV FRS 13299). The three dose rates of ISAV used to seed the tubes were 1000, 5000 or 25,000 TCID₅₀.

The tubes were incubated at 15° C. for 90 minutes. The buffy coat tubes were centrifuged to pellet the cells and all medium was removed. The cells were then resuspended in fresh growth medium and the tubes were incubated at 15° C. for ten days. Samples were taken at day 3 and 10 and analysed for ISAV growth using quantitative PCR (qPCR). All samples were tested in triplicate.

The qPCR was a TaqMan assay. Primers were ISAV S7-F1 (5′-TGG GAT CAT GTG TTT CCT GCT A-3′ (SEQ ID NO:4)) and ISAV S7-R1 (5′-GAA AAT CCA TGT TCT CAG ATG CAA-3′ (SEQ ID NO:5)). The TaqMan probe used was ISAV S7-probe (5′-6FAM CAC ATG ACC CCT CGT C MGBNFQ-3′ (SEQ ID NO:6)). The qPCR has been described previously (Plarre et al., 2005).

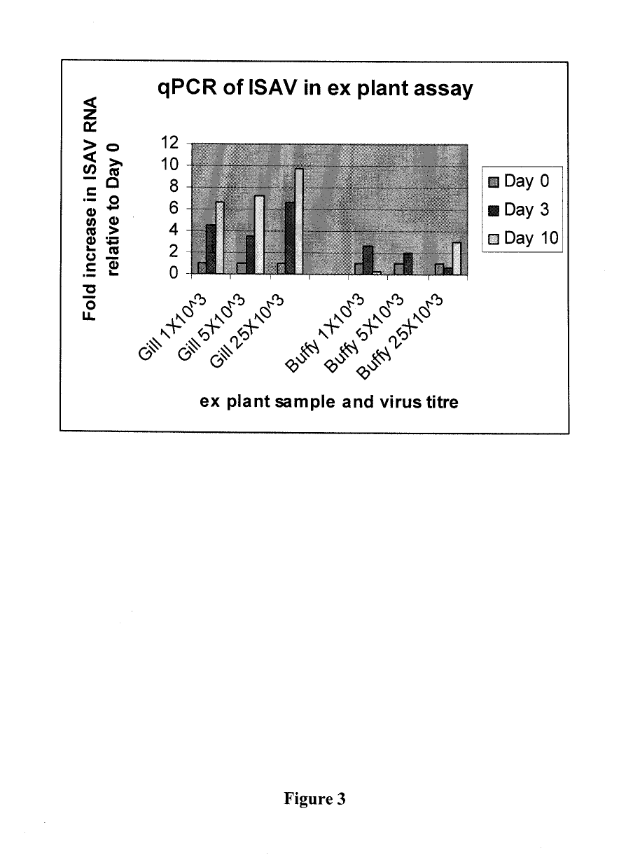

qPCR results are shown in FIG. 3. ISAV was able to grow in the gill explant samples, but was unable to grow in the buffy coat samples. In the gill explant samples, virus growth was detected at day 3 and day 10 for all three virus doses described above. The greatest amount of virus growth was detected in the Day 10 gill explant sample using a dose of 25,000 TCID₅₀. At this dose, ISAV RNA was almost 10 fold higher at Day 10 compared with Day 0.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

The present application claims priority from U.S. 61/218,742 filed 19 Jun. 2009, the entire contents of which are incorporated herein by reference.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Bonneau, K R et al. (2001) 75:8298-8305.
Burkhardt E and Muller H (1987) Arch Virol. 94:297-303.
Christopher-Hennings J et al. (1995) J Vet Diag Invest. 7:456-464.
Collins J E et al. (1992) J Vet Diagn Invest. 4:117-126.
Cosgrove A S (1962) Avian Dis. 6:385-389.
Falk, et al. (1997) J Virol. 71:9016-23.
Heine H et al. (2007) Avian Dis. 51(1 Suppl):370-372.
Kibenge F S et al. (1988) J Gen Virol. 69:1757-1775.
Kwoh D Y et al. (1989) Proc Natl Acad Sci USA. 86:1173-1177.
Lasher H N and Davis V S (1997) Avian Dis. 41:11-19.
Lawson S R et al. (1997) Virus Res. 51:105-113.
McNulty M S et al. (1990) Avian Pathol. 19:67-73.
Notomi T et al. (2000) Nucleic Acids Res. 28:E63.
Plarre et al. (2005) Dis Aquat Org, 66:71-79.
Spreull J (1905) J Comp Path Ther. 18:321-337.
van den Berg et al. (2000) Rev Sci Tech. 19:509-543.
Vielitz E et al. (1989) Poult Sci. 68:34-35.
von Bulow V et al. (1983) Zentralbl Veterinarmed B. 30:742-750.
Walker G T et al. (1992a) Proc Natl Acad Sci USA. 89:392-396.
Walker G T et al. (1992b) Nucleic Acids Res. 20:1691-1696.
Wensvoort G et al. (1991) Vet Q, 13:121-130.
Wilson W C et al. (2000) Virus Res. 62:141-151.
Yuasa N et al. (1979) Avian Dis. 23:366-385.

1. A method for determining the susceptibility of a subject to a virus and/or detecting viral replication in a tissue sample from a subject, the method comprising:
contacting a tissue sample obtained from the subject with the virus,
incubating the tissue sample for time sufficient for viral replication, and
detecting the presence or absence of virus in the tissue sample.

2. (canceled)

3. The method of claim 1, further comprising removing virus that is not attached to a cell in the tissue sample prior to incubating the sample for time sufficient for viral replication.

4. The method of claim 1, wherein the presence of virus is indicative of susceptibility to the virus.

5. The method of claim 1, further comprising comparing the level of viral replication in the sample with a control sample.

6. The method of claim 5, wherein a decreased level of virus in the tissue sample compared to the control sample is indicative of a decreased susceptibility to the virus.

7. The method of claim 1, wherein the subject is avian.

8-11. (canceled)

12. The method of claim 1, wherein the subject is a fish.

13-14. (canceled)

15. The method of claim 1, wherein detecting the presence or absence of virus in the tissue sample comprises isolating nucleic acid from the tissue sample.

16-17. (canceled)

18. The method of claim 1, wherein the virus is selected from influenza virus, Newcastle disease virus, chicken anaemia virus, infectious bursal disease virus, foot and mouth disease virus, porcine reproductive and respiratory syndrome virus, classical swine fever virus, bluetongue virus, akabane virus, infectious salmon anemia virus, infectious hematopoietic necrosis virus, viral haemorrhagic septicaemia virus and infectious pancreatic necrosis virus.

19-39. (canceled)

40. The method of claim 1, wherein the tissue sample comprises transgenic cells.

41. The method of claim 40, wherein the transgenic cells comprise a transgene encoding a dsRNA molecule.

42. The method of claim 41, wherein the dsRNA molecule is an shRNA molecule.

43. The method of claim 41, wherein the dsRNA molecule comprises a viral nucleic acid sequence.

44. (canceled)

45. A method for identifying an animal having decreased susceptibility to a virus, the method comprising:
(i) performing the method of claim 1, and
(ii) identifying animals having decreased susceptibility to a virus.

46. A method for breeding animals, the method comprising:
(i) performing the method of claim 1;
(ii) selecting an animal having decreased susceptibility to a virus; and
(iii) breeding from the animal.

47. The method of claim 46, further comprising:
(i) selecting a first animal of a first gender having decreased susceptibility to the virus; and
(ii) selecting a second animal of the opposite gender having decreased susceptibility to the virus; and
(iii) mating the first and second animals to produce offspring.

48. A kit for performing the method of claim 1, the kit comprising means for detecting the virus.

49. The kit of claim 48, comprising at least one nucleic acid molecule which hybridises under stringent conditions with a viral nucleic acid.

50. The kit of claim 49, wherein the at least one nucleic acid molecule is a primer useful for amplifying a viral nucleic acid.

51. The kit of claim 48 further comprising a sample of virus.


SEQUENCE LISTING (13 sequences)

SEQ ID NO: 1 - DNA, 1027 bp, Influenza A virus
agcgaaagcaggtagatattgaaagatgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggggtgcagatgcaacggttcaagtgatcctctcactattgccgcaaatatcattgggatcttgcacttgacattgtggattcttgatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaaaaaactaccttgtttctact

SEQ ID NO: 2 - DNA, 101 bp, Artificial
  M gene amplicon
agatgagtcttctaaccgaggtcgaaacgtacgtactctctatcatcccgtcaggccccctcaaagccgagatcgcacagagacttgaagatgtctttgca

SEQ ID NO: 3 - DNA, 19 bp, Artificial Sequence
  PB1 sequence targeted by shRNA
gatctgttccaccattgaa

SEQ ID NO: 4 - DNA, 22 bp, Artificial Sequence
  Oligonucleotide primer
tgggatcatgtgtttcctgcta

SEQ ID NO: 5 - DNA, 24 bp, Artificial Sequence
  Oligonucleotide primer
gaaaatccatgttctcagatgcaa

SEQ ID NO: 6 - DNA, 16 bp, Artificial Sequence
  Fluorescent probe
cacatgacccctcgtc

SEQ ID NO: 7 - DNA, 25 bp, Artificial Sequence
  Oligonucleotide primer
cgacgatgactctctactgtgtgat

SEQ ID NO: 8 - DNA, 20 bp, Artificial Sequence
  Oligonucleotide primer
tcatcagtgtcgccatgctt

SEQ ID NO: 9 - DNA, 14 bp, Artificial Sequence
  Fluorescent probe
acggtggatctttc

SEQ ID NO: 10 - DNA, 966 bp, Infectious salmon anemia virus
tacaaagaaaatgttcagaacatgtctggatttaacttcgaggtaatggtgccggaacaaggaggaaaagtggtcttcagccttactgaaacggggtcatgtgtctcgttttacggagatgatgaaccaggtgaagggtcctgcgaacttgcctctgaaaacatggattttccaagttgtcctctggggaatggagatgacttctgtctgtcgctggcgctaagcacaatgagatggtctgggatgaccaagagaaacaacttcatggacagattcattggaagttttgttcattgtacaccagtgatgatctggtcgtatggaaatttgtccaagaaaagccatcacaaaatggtttgccacacttgcccagacgagtacaagttcagtgacaaggacgagatgcagggatactatgaggaatgtctagaggcttctactgacattttccttgatgaacttgctactgttgttacaggtggcttctttcctgtcggactcaaaggttcctggggaggatggtacctcaagtacgtcaggtatgctggacctcttgcgggatcaagtggattcattgtcaatcaacgattctacgacagagcccaaaacaagactggatccagggttgtatccatggttgaaatggacggagacggcttatcgttcatctacgagaagcctagcgtctaccatagtgatgggtgcactggttcagcagcgaggttctggaaacgggatcacaatgagagagctggagttgagcttagggctggacttcacttcagaatgtgattggttgaaaacttgttatgtaaacaagaattttgtgtttttgtcagaaaaagaaattgctgtaaacatggaagttgaaaaattcatttgtaatgagaactaaagatgtctttgtgttcaaattttaactaatgacaatatatgaaatatgtcgtacatggtgttgatgataat

SEQ ID NO: 11 - DNA, 736 bp, Infectious salmon anemia virus
tgcaaagattggctatctaccatgcatgagagaagcaaacccaaaaccacgggagctgatcagacatgccttgaagaagaaaaagagaccagaggtggtttacgcaatgggagttcttctgacactggggggagagagcggactgaccgtggagtttcctgttccagaaggaaaaactgtgaaggtcaaaaccttgaaccaattggtgaacgggatgatcagtcgagcgacgatgaccctctactgtgtgatgaaagatccaccatcgggaggcatggcaacgctgatgagagaccacatcaggaactggctgaaggaggaatcaggatgccaggacgcggatggtggagaggaaaaatgggcaatggtgtatggtatgatttcacccgacatggcagaggagaagacgatgctgaaggagctgaaaacaatgctacacagcaggatgcagatgtatgctctgggtgcaagttcgaaagccctagagaatttagaaaaggccatcgtcgctgcagttcatcgacttccggcatcctgctcgacagagaagatggtgcttctggggtacctgaagtaagcttcaaagaaagaatggaagcggagaagaagaaactgaaagagctggacgacaagatctacaagctaaggagaagattgaggaagatggagtacaagaaaatggggatcaaccgagaaatcgacaaattggaagactctgtacaataaaatcactagt

SEQ ID NO: 12 - DNA, 81 bp, Infectious salmon anemia virus
tgggatcatgtgtttcctgctacggggacgacgaacctgacgaggggtcatgtgaacttgcatctgagaacatggattttc

SEQ ID NO: 13 - DNA, 63 bp, Infectious salmon anemia virus
cgacgatgactctctactgtgtgatgaaagatccaccgtctggaagcatggcgacactgatga